(19) United States
(12) Patent Application Publication
(10) Pub. No.: US 2023/0270896 A1
(43) Pub. Date: Aug. 31, 2023

(54) SYSTEM AND METHOD OF ELIMINATING MICROORGANISMS

(71) Applicant: NATIONAL UNIVERSITY OF SINGAPORE, Singapore (SG)

(72) Inventors: Weibiao ZHOU, Singapore (SG); Vinayak GHATE, Singapore (SG); Ye Htut ZWE, Singapore (SG); Hyun-Gyun YUK, Chungbuk (KR)

(73) Assignee: NATIONAL UNIVERSITY OF SINGAPORE, Singapore (SG)

(21) Appl. No.: 17/780,697

(22) PCT Filed: Nov. 27, 2020
(86) PCT No.: PCT/SG2020/050701

§ 371 Date: May 27, 2022

(30) Foreign Application Priority Data
Nov. 27, 2019 (SG) 10201911270R

(51) Int. Cl. A61L 2/10; A61L 2/26; A61L 2/08

(52) U.S. Cl. CPC A61L 2/10; A61L 2/26; A61L 2/084; A61L 2202/11; A47K 13/302

(57) ABSTRACT

The present invention provides a system for eliminating microorganisms comprising a plurality of light emitters, wherein each of the plurality of light emitters is configured to emit light comprising a wavelength of 380-500 nm, and wherein at least two or more of the plurality of light emitters emit light of a different wavelength. There is also provided a method of eliminating microorganisms comprising emitting a light of a wavelength of 380-500 nm by each of a plurality of light emitters.

### CROSS-REFERENCE TO RELATED APPLICATIONS

This application is a National Stage Patent Application (filed under 35 § U.S.C. 371) of PCT/SG2020/050701, of the same title filed Nov. 27, 2020, which, in turn claims priority to SG10201911270R titled “A METHOD TO PRODUCE AN ANTIMICROBIAL EFFECT BY OPTIMALLY EXCITING A VARIETY OF PHOTOSENSITIZERS IN A MICROBIAL CELL” filed Nov. 27, 2019; the contents of each of which are hereby incorporated by reference.

### TECHNICAL FIELD

The present invention relates to a system and method of eliminating microorganisms.

### BACKGROUND

Microorganisms exist everywhere around us and with each increase in diseases and infections spread by microorganisms, more efficient disinfection methods are always being explored. Existing disinfection methods such as ultraviolet and hydrogen peroxide vapors can only be used episodically. This may be due to safety concerns or the need for manual effort.

In view of the safety concerns, the option of using visible light has been explored. The visible light spectrum as an alternative or supplementary disinfecting method allows continuous disinfection due to the fact that it is completely safe to humans. However, existing methods of using visible light to kill microorganisms involve the use of a single wavelength, most commonly at a peak wavelength of 405 nm, which is only able to excite a portion of photosensitizer comprised in microorganisms, which have an absorption peak of 405 nm. Accordingly, complete elimination of microorganisms may not be achieved.

Therefore, there is a need for an improved system and method of eliminating microorganisms.

### SUMMARY OF THE INVENTION

The present invention seeks to address these problems, and/or to provide an improved system and method of eliminating microorganisms.

According to a first aspect, the present invention provides a system for eliminating microorganisms, the system comprising a plurality of light emitters, wherein each of the plurality of light emitters is configured to emit light comprising a wavelength of 380-500 nm, and wherein at least two or more light emitters of the plurality of light emitters emit light of a different wavelength. In particular, the plurality of light emitters may be configured to emit light comprising a wavelength of 380-430 nm.

According to a particular aspect, each of the plurality of light emitters may be configured to emit light of a different wavelength from one another.

For example, the plurality of light emitters may comprise at least two light emitters. In particular, the plurality of light emitters may comprise at least three light emitters.

According to a particular aspect, the system may comprise three light emitters, such that each of the three light emitters is configured to emit light of a wavelength of 385-395 nm, 395-405 nm and 405-425 nm, respectively. In particular, each of the three light emitters may be configured to emit light of a wavelength of 390 nm, 405 nm and 425 nm, respectively.

The microorganism may be any suitable microorganism. For example, the microorganism may be bacteria, yeasts, molds, algae, or a combination thereof. In particular, the microorganisms may be eliminated upon illumination with light emitted by the plurality of light emitters.

The light emitter may be any suitable light emitter. For example, the light emitter may comprise a light-emitting diode (LED), an organic light-emitting diode (OLED), a semiconductor die, laser, incandescent lamp, superluminescent diode (SLD), or a combination thereof.

The present invention also provides a method of eliminating microorganisms, the method comprising emitting a light comprising a wavelength of 380-500 nm by each of a plurality of light emitters. The microorganisms may be eliminated upon illumination with light emitted by the plurality of light emitters. In particular, the emitting may comprise emitting a light comprising a wavelength of 380-430 nm by each of a plurality of light emitters.

The microorganism may be as described above in relation to the first aspect. The light emitter may be as described above in relation to the first aspect.

According to a particular aspect, each of the plurality of light emitters may emit light of a different wavelength from each of the other light emitters of the plurality of light emitters.

The plurality of light emitters may comprise at least two light emitters. In particular, the plurality of light emitters may comprise at least three light emitters.

According to a particular aspect, the plurality of light emitters may comprise three light emitters, such that each of the three light emitters emits light of a wavelength of 385-395 nm, 395-405 nm and 405-425 nm, respectively. In particular, each of the three light emitters emits light of a wavelength of 390 nm, 405 nm and 425 nm, respectively.

### BRIEF DESCRIPTION OF THE DRAWINGS

In order that the invention may be fully understood and readily put into practical effect there shall now be described by way of non-limitative example only exemplary embodiments, the description being with reference to the accompanying illustrative drawings. In the drawings:

FIG. 1 shows a schematic representation of a device incorporating the system for eliminating microorganisms according to one embodiment of the present invention;

FIG. 2(A) shows a schematic representation of the effect of emitting light of a single wavelength on photosensitizer excitation and FIG. 2(B) shows a schematic representation of the effect of emitting light of a combination of wavelengths on photosensitizer excitation;

FIG. 3 shows a cross-sectional representation of a visible light disinfection system;

FIG. 4 shows a schematic representation of a visible light disinfection system utilizing a combination of wavelengths of light;

FIG. 5 shows inactivation plot of E. coli (ATCC 25922) following illumination with different combinations of wavelengths of light and with light of wavelength of 405 nm only;

FIG. 6 shows a schematic representation of a device incorporating the system for eliminating microorganisms according to one embodiment of the present invention; and

FIG. 7 shows inactivation plot of E. coli (ATCC 25922) following illumination with a combination of wavelengths of light and without any light.

### DETAILED DESCRIPTION

As explained above, there is a need for an improved disinfection system and method for eliminating microorganisms.

In general terms, the present invention relates to a system and method for killing microorganisms by using the visible light spectrum to optimally excite a variety of endogenous photosensitizers in a microbial cell. In particular, the present invention provides a system and method for possibly continuously disinfecting surfaces to enable effective elimination of microorganisms on surfaces.

According to a first aspect, the present invention provides a system for eliminating microorganisms, the system comprising a plurality of light emitters, wherein each of the plurality of light emitters is configured to emit light comprising a wavelength of 380-500 nm, and wherein at least two or more light emitters emit light of a different wavelength.

In particular, each of the plurality of light emitters is configured to emit light comprising a wavelength of 380-430 nm. For example, each of the plurality of light emitters is configured to emit light comprising a wavelength of 380-450 nm, 390-430 nm, 395-425 nm, 400-420 nm, 405-415 nm, 410-412 nm.

The microorganism may be any suitable microorganism. For example, the microorganism may be, but not limited to, bacteria, yeasts, molds, algae, or a combination thereof. In particular, the microorganisms may be eliminated upon illumination with light emitted by the plurality of light emitters.

Microorganisms may comprise light sensitive photosensitizers, such as, but not limited to, porphyrins. Upon excitation, these photosensitizers may trigger cell death through a series of cytotoxic reactions via the generation of reactive oxygen species (ROS) such as singlet oxygen, hydroxyl radical, hydrogen peroxide, or superoxide anion. These ROS subsequently cause cytotoxic reactions inside the microorganisms that ultimately lead to death of the microorganism. Each photosensitizer may have a unique absorption spectra and each microorganism has a different composition of these compounds.

By providing a plurality of light emitters configured to emit light comprising a wavelength of 380-500 nm, and with at least two or more light emitters of the plurality of light emitters being configured to emit light of a different wavelength, in use, a different composition of ROS may be generated since different photosensitizers may be excited at different wavelengths. In this way, a wide variety of endogenous photosensitizers may be excited and consequently, a different composition of ROS may be generated by targeting multiple porphyrin compounds in a microorganism as opposed to a single porphyrin when light of only one wavelength is emitted. Accordingly, the antimicrobial effect may be enhanced.

For the purposes of the present invention, optimal excitation may be defined as the absorption peak of a photosensitizer coinciding with the emission peak of the lighting element within the visible light electromagnetic spectrum.

According to a particular aspect, each of the plurality of light emitters may be configured to emit light of a different wavelength from one another. In this way, more photosensitizers may be excited and more ROS may be generated to bring about cell death in microorganisms.

The plurality of light emitters may comprise any suitable number of light emitters. For example, the plurality of light emitters may comprise at least two, at least three, at least four light emitters. In particular, at least two, three or four of the plurality of the light emitters may be configured to emit light of a different wavelength, wherein the light comprises a wavelength of 380-500 nm, particularly wherein the light comprises a wavelength of 380-430 nm.

According to a particular aspect, the system may comprise three light emitters, such that each of the three light emitters is configured to emit light of a different wavelength. For example, the first light emitter may be configured to emit light of a wavelength of 385-395 nm, the second light emitter may be configured to emit light of a wavelength of 395-405 nm and the third light emitter may be configured to emit light of a wavelength of 405-425 nm, respectively. In particular, the first light emitter may be configured to emit light of a wavelength of 385-395 nm, the second light emitter may be configured to emit light of a wavelength of 395-405 nm and the third light emitter may be configured to emit light of a wavelength of 405-415 nm, respectively. According to a particular embodiment, each of the three light emitters may be configured to emit light of a wavelength of 390 nm, 405 nm and 425 nm, respectively. According to another embodiment, each of the three light emitters may be configured to emit light of a wavelength of 395 nm, 405 nm and 415 nm, respectively. In another embodiment, each of the three light emitters may be configured to emit light of a wavelength of 385 nm, 395 nm and 405 nm, respectively.

The light emitter may be any suitable light emitter. For example, the light emitter may comprise a light-emitting diode (LED), an organic light-emitting diode (OLED), a semiconductor die, laser, incandescent lamp, superluminescent diode (SLD), or a combination thereof. In particular, the light emitter may be an LED.

The plurality of light emitters may be configured to emit light of a suitable radiance. For example, the radiance may be 0.001-1000 mW/cm². In particular, the radiance may be 0.01-800 mW/cm², 0.1-600 mW/cm², 0.5-500 mW/cm², 1-300 mW/cm², 5-250 mW/cm², 10-200 mW/cm², 40-175 mW/cm², 50-150 mW/cm², 75-100 mW/cm². Even more in particular, the radiance may be 10-40 mW/cm².

The system of the present invention may be used in any suitable device. For example, the system may be used in devices which require frequent or constant disinfecting. According to a particular aspect, at least part of the device may be transparent to enable the light emitted from the light emitters to reach a surface of the device where disinfection is required. For example, the device may be a toilet seat, a toilet bowl, a door knob, a door handle, or any device or surface which is of high touch frequency.

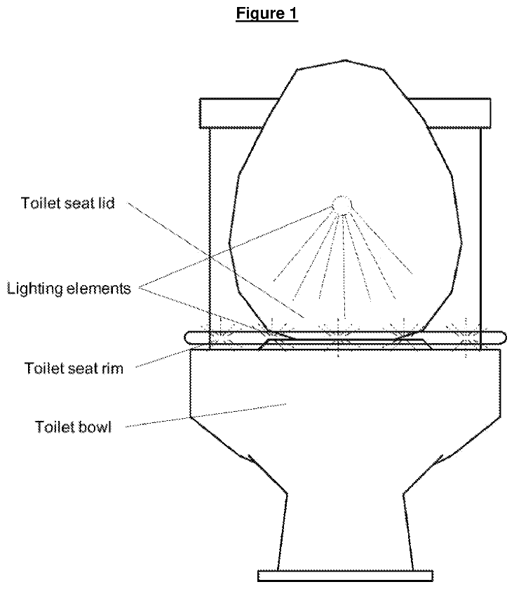

According to a particular aspect, the system may be used in a toilet seat. The toilet seat, or part of the toilet seat, incorporating the system described above may be transparent. In particular, the system of the present invention, in use, may illuminate light from within a transparent seating rim which disinfects the surface of the toilet seat that comes into contact with the users. An additional system illuminating the disinfecting light may be attached to the lid of the toilet seat, such that when the lid is closed, the system may deliver disinfecting effect onto the toilet bowl below. An example of such a toilet bowl is shown in FIG. 1.

In particular, the toilet seat rim which the user generally sits on may be made of a transparent material, such as, but not limited to, polyresin. Within the body of the rim, a single lighting element powered by a main supply which features the combination of wavelength as described above may run through the perimeter of the rim. The transparent nature of the material allows the light emitted from the lighting element embedded within to reach the surface of the rim and decontaminate it.

The toilet seat may also include a lid at the center of which features a light source. When the lid is closed, the light source may be configured to emit light which will disinfect the inner surface of the toilet bowl.

The present invention also provides a method of eliminating microorganisms by using the system described above.

According to a second aspect, the present invention provides a method of eliminating microorganisms, the method comprising emitting a light comprising a wavelength of 380-500 nm by each of a plurality of light emitters, wherein at least two or more light emitters emit light of a different wavelength. The microorganisms may be eliminated upon illumination with light emitted by the plurality of light emitters.

In particular, the emitting may comprise emitting a light comprising a wavelength of 380-430 nm by each of the plurality of light emitters. For example, the emitting may comprise emitting a light comprising a wavelength of 380-450 nm, 390-430 nm, 395-425 nm, 400-420 nm, 405-415 nm, 410-412 nm by each of the plurality of light emitters.

The microorganism may be as described above in relation to the first aspect.

The light emitter may be as described above in relation to the first aspect.

The plurality of light emitters may comprise any suitable number of light emitters. For example, the plurality of light emitters may comprise at least two, at least three, at least four light emitters. In particular, at least two, at least three or at least four of the plurality of the light emitters may emit light of a different wavelength, wherein the light comprises a wavelength of 380-430 nm.

According to a particular aspect, each of the plurality of light emitters may emit light of a different wavelength from one another.

According to one embodiment, the plurality of light emitters may comprise three light emitters, such that each of the three light emitters may emit light of a different wavelength. For example, the first light emitter may emit light of a wavelength of 385-395 nm, the second light emitter may emit light of a wavelength of 395-405 nm, and the third light emitter may emit light of a wavelength of 405-425 nm. In particular, the first light emitter may emit light of a wavelength of 385-395 nm, the second light emitter may emit light of a wavelength of 395-405 nm and the third light emitter may emit light of a wavelength of 405-415 nm, respectively. According to a particular embodiment, each of the three light emitters may emit light of a wavelength of 390 nm, 405 nm and 425 nm, respectively. According to another embodiment, each of the three light emitters may emit light of a wavelength of 395 nm, 405 nm and 415 nm, respectively. In another embodiment, each of the three light emitters may emit light of a wavelength of 385 nm, 395 nm and 405 nm, respectively.

The emitting may comprise the plurality of light emitters emitting light of a suitable radiance. For example, the radiance may be 0.001-1000 mW/cm². In particular, the radiance may be 0.01-800 mW/cm², 0.1-600 mW/cm², 0.5-500 mW/cm², 1-300 mW/cm², 5-250 mW/cm², 10-200 mW/cm², 40-175 mW/cm², 50-150 mW/cm², 75-100 mW/cm². Even more in particular, the radiance may be 10-40 mW/cm².

Microorganisms contain a variety of photosensitizers such as, but not limited to, flavins, porphyrins, chlorophylls, bilirubin. Each of these compounds has a different absorption spectrum. For example, the absorption spectrum of riboflavin in the visible region has a peak at 446.5 nm while coproporphyrin has its peak at 390 nm, while numerous other forms of compounds from the porphyrin family have absorption peaks ranging from 415 to 425 nm. The excitation of different groups of photosensitizers also generate different compositions of ROS.

Existing methods of using visible light to kill microorganisms involve the use of one wavelength, mostly 405 nm, which can only optimally excite the photosensitizer with a corresponding absorption peak spectrum of 405 nm while other portions of endogenous photosensitizers within the microorganism cell will not be optimally excited for the killing effect. This is schematically shown in FIG. 2A. For example, a single lighting element with an absorption peak of 405 (±5) nm will not efficiently excite the photosensitizer coproporphyrin I which has a peak absorbance of 390 nm and comparatively much less absorbance at 405±5 nm.

In view of this limitation, the method of the present invention uses a combination of wavelengths which will ensure that multiple endogenous photosensitizers from the porphyrin group that may be present in the microorganism is excited simultaneously, as shown in FIG. 2B. Excitation of multiple photosensitizers may lead to the generation of different compositions of ROS, which will enhance the efficacy of the subsequent cytotoxic reactions. This represents a marked improvement over prior art methods of visible light disinfection methods.

### Example 1

Bacterial Culture Conditions

Escherichia coli ATCC 25922 was cultured in tryptone soya broth (TSB) at 37° C. for 24 h at least twice before every experiment. A 1 mL portion of the bacterial suspension was centrifuged at 9000×g for 5 min, washed twice with phosphate buffer saline (PBS) and serially diluted in 0.1% peptone water (PW) to approximately 106 CFU/mL cell density in 0.1% PW. A 5 mL portion of this bacterial suspension was dispensed into a 6 cm diameter petri dish for visible light disinfection treatment.

Visible Light Disinfection (VLD) System and Configuration

A total of three configurations of disinfection devices were built:
1) 405 nm single wavelength (λ) control;
2) Standard λ combination (395 nm+405 nm+415 nm); and
3) Low λ combination (385 nm+395 nm+405 nm).

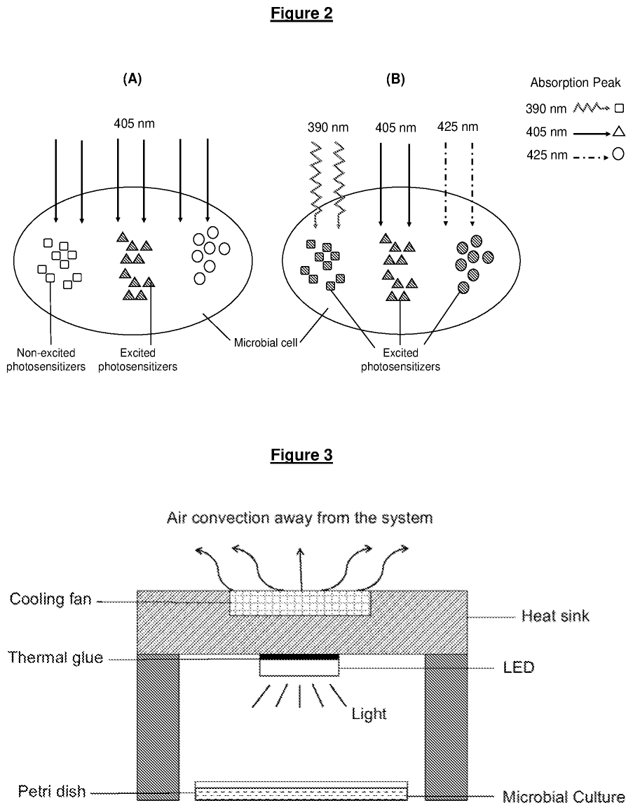

The device used in configuration 1 is as shown in FIG. 3, featuring a heatsink with cooling fan and a single 405 nm LED light attached at the center.

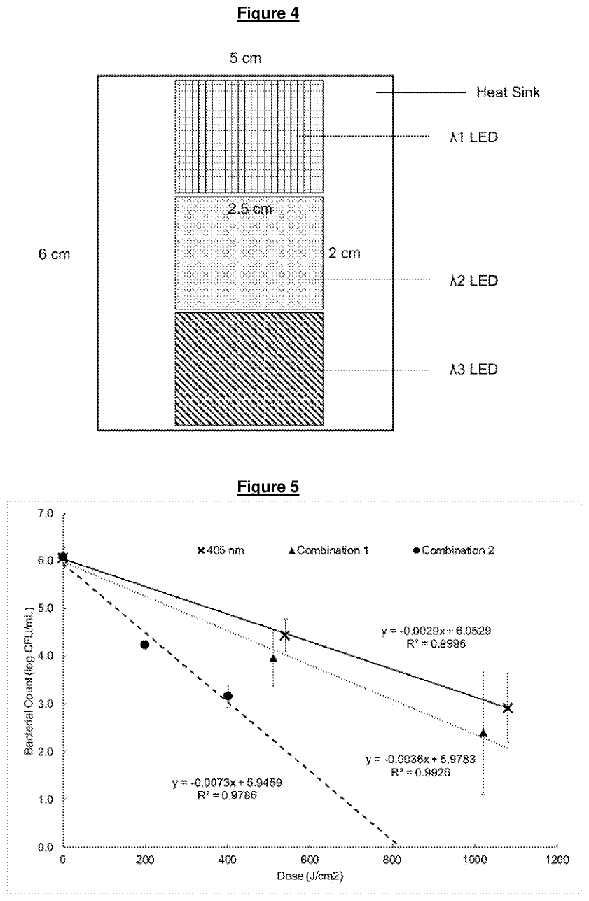

The devices used for configurations 2 and 3 were similarly built, but each with 3 LEDs of different λs as dictated by the respective configuration in place of a single LED (as shown in FIG. 4)

VLD Treatment

The set-up as depicted in FIG. 3 was used for the VLD treatment of the 5 mL portion bacterial suspension prepared. A total of 4 such petri dish samples were set up and each was exposed to VLD treatment with either configuration 1, 2, or 3 while the 4th petri dish was set up as a non-illuminated control. At 45 min intervals up to 3 h, a 100 μL aliquot was drawn from each sample and bacterial count was enumerated by plating on tryptone soya agar (TSA) following appropriate serial dilution in 0.1% PW. The TSA plates were incubated at 37° C. for 24 h.

Results and Analysis

The bacterial count at different time points were expressed as bacterial count at different doses of light energy using the following formula:

$$Dose ( \frac{J}{sq . cm} ) = Irradiance ( \frac{W}{sq . cm} ) \times time ( s )$$

Irradiance (1) was experimentally measured using PM 100D Optical Power Meter (ThorLabs). For configuration 1, I₄₀₅ was measured while effective I for configurations 2 and 3 were calculated as follows:
Configuration 2: Iₑ=(I₃₉₅+I₄₀₅+I₄₁₅)÷3
Configuration 3: Iₑ=(I₃₈₅+I₃₉₅+I₄₀₅)÷3

A scatter plot of bacterial count against dose was plotted and the D-value (decimal reduction time) for each configuration was calculated as the negative reciprocal of the gradient of the trendline. Table 1 shows the D-value for each configuration while FIG. 5 shows the inactivation plots.

TABLE 1
Decimal reduction time of E. coli (ATCC 25922)
upon illumination by different setups

| Illumination Mode | D-value (J/cm²) |
|---|---|
| Configuration 1 | 344.8 ± 66.5 |
| Configuration 2 | 277.8 ± 36.7 |
| Configuration 3 | 137.0 ± 16.2 |

It can be seen from FIG. 5 that by using a combination of wavelengths, significantly better inactivation/elimination of microorganism was obtained as compared to when a single wavelength of light was used for the disinfection.

### Example 2

Bacterial Culture Conditions

Escherichia coli ATCC 25922 was cultured in tryptone soya broth (TSB) at 37° C. for 24 h at least twice before every experiment. A 1 mL portion of the bacterial suspension was centrifuged at 9000×g for 5 min, washed twice with phosphate buffer saline (PBS) and serially diluted to obtain bacterial suspension of approximately 10⁷ CFU/mL cell density in TSB.

Self-Disinfecting Door Handle

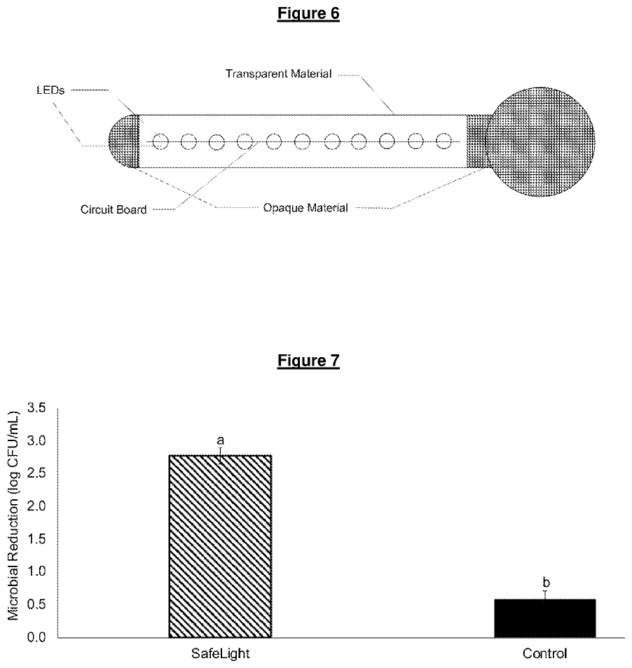

A prototype of a door handle consisting of a transparent hollow acrylic tube with two lighting modules attached back-to-back inserted was constructed as shown in FIG. 6. Each lighting module comprised LED bulbs of different peak wavelengths.

VLD Treatment

A total of 10 spots of 10 μL, portions each of the bacterial suspension (approximately 10⁶ CFU total) described above was deposited on the length of the acrylic tube. An identical door handle design without lighting module inserted was also inoculated with bacterial suspension as control. The door handle was illuminated for 2 h and the surface was swabbed, and the bacterial count was enumerated on tryptone soya agar (TSA) incubated at 37° C. for 24 h. Antimicrobial effect was determined from the reduction of bacterial count from inoculated levels at 0 h to that at 2 h in control and illuminated door handles.

Results

As shown in FIG. 7, the VLD treatment (termed SafeLight) shows significant microbial reduction as compared to the control, thereby showing that the VLD treatment according to the present invention is effective in eliminating microorganisms on surfaces.

While the foregoing description has described exemplary embodiments, it will be understood by those skilled in the technology concerned that many variations may be made without departing from the present invention. Further, the exemplary embodiments are only examples, and are not intended to limit the scope, applicability, operation or configuration of the invention in any way.

1. A system for eliminating microorganisms, the system comprising a plurality of light emitters, wherein each of the plurality of light emitters is configured to emit light comprising a wavelength of 380-500 nm, and wherein at least two of the plurality of light emitters emit light of a different wavelength.

2. The system of claim 1, wherein each of the plurality of light emitters is configured to emit light comprising a wavelength of 380-430 nm.

3. The system of claim 1, wherein each of the plurality of light emitters is configured to emit light of a different wavelength from each of the other light emitters of the plurality of light emitters.

4. The system of claim 1, wherein the system comprises at least two light emitters.

5. The system of claim 1, wherein the system comprises at least three light emitters.

6. The system of claim 1, wherein the system comprises three light emitters, such that each of the three light emitters is configured to emit light of a wavelength of 385-395 nm, 395-405 nm and 405-425 nm, respectively.

7. The system of claim 1, wherein the system comprises three light emitters, such that each of the three light emitters is configured to emit light of a wavelength of 390 nm, 405 nm and 425 nm, respectively.

8. The system of claim 1, wherein the microorganism is bacteria, yeast, mold, algae, or a combination thereof.

9. The system of claim 1, wherein the microorganisms are eliminated upon illumination with light emitted by the plurality of light emitters.

10. The system of claim 1, wherein the plurality of light emitters comprises a light-emitting diode (LED), an organic light-emitting diode (OLED), a semiconductor die, laser, incandescent lamp, superluminescent diode (SLD), or a combination thereof.

11. A method of eliminating microorganisms, the method comprising emitting a light comprising a wavelength of 380-500 nm by each of a plurality of light emitters.

12. The method of claim 11, wherein the emitting comprises emitting a light comprising a wavelength of 380-430 nm by each of the plurality of light emitters.

13. The method of claim 11, wherein each of the plurality of light emitters emits light of a different wavelength from one another each of the other light emitters of the plurality of light emitters.

14. The method of claim 11, wherein the plurality of light emitters comprises at least two light emitters.

15. The method of claim 11, wherein the plurality of light emitters comprises at least three light emitters.

16. The method of claim 11, wherein the plurality of light emitters comprises three light emitters, such that each of the three light emitters emits light of a wavelength of 385-395 nm, 395-405 nm and 405-425 nm, respectively.

17. The method of claim 11, wherein the plurality of light emitters comprises three light emitters, such that each of the three light emitters emits light of a wavelength of 390 nm, 405 nm and 425 nm, respectively.

18. The method of claim 11, wherein the microorganism is bacteria, yeast, mold, algae, or a combination thereof.

19. The method of claim 11, wherein the microorganisms are eliminated by the emitting of the light.

20. The method of claim 11, wherein the plurality of light emitters comprises a light-emitting diode (LED), an organic light-emitting diode (OLED), a semiconductor die, laser, incandescent lamp, superluminescent diode (SLD), or a combination thereof.
